(19) United States
(12) Patent Application Publication
(10) Pub. No.: US 2016/0174837 A1
(43) Pub. Date: Jun. 23, 2016

(54) METHOD FOR MEASURING TWO PUPILS AND DUAL-PUPIL MEASURING DEVICE

(71) Applicants: NATIONAL CHIAO TUNG UNIVERSITY, Hsinchu City (TW); National Taiwan University Hospital Hsin-Chu Branch, Hsinchu City (TW)

(72) Inventors: Mang OU-YANG, Hsinchu City (TW); Ting-Wei HUANG, Taipei City (TW); Jin-Chern CHIOU, Hsinchu City (TW); Mei-Lan KO, Hsinchu City (TW); Bak-Sau YIP, Hsinchu City (TW); Chih-Cheng WU, Hsinchu City (TW); Wei-De JENG, Taoyuan City (TW); Yin-Yuan CHEN, Changhua County (TW); Bi-Shou SONE, Taoyuan City (TW)

(21) Appl. No.: 14/686,759

(22) Filed: Apr. 14, 2015

(30) Foreign Application Priority Data
Dec. 23, 2014 (TW) 103145029

(51) Int. Cl. A61B 3/11; A61B 3/14

(52) U.S. Cl. CPC A61B 3/112; A61B 3/14

(57) ABSTRACT

A method for measuring two pupils includes a number of steps. A first visible light beam and a first invisible light beam are emitted toward a right eye. A second visible light beam and a second invisible light beam are emitted toward a left eye. The light beams reflected from the right eye and the left eye are received by an optical unit, and the first invisible light beam and the second invisible light beam are guided to an imaging unit by the optical unit. Images of the right eye and the left eye are respectively recorded through the first invisible light and the second invisible light beam propagated from the optical unit.

### RELATED APPLICATIONS

This application claims priority to Taiwan Application Serial Number 103145029, filed Dec. 23, 2014, which is herein incorporated by reference.

### BACKGROUND

1. Field of Invention

The present invention relates to a pupil measuring device and a method for measuring a pupil. More particularly, the present invention relates to a dual-pupil measuring device and a method for measuring two pupils.

2. Description of Related Art

With developments in the medical field and in technology, there have also been developments in the area of health examinations. For some potential problems that are difficult to detect in the human body, the human body may be stimulated using a specific instrument, such that a possible cause of a disease can be found by analyzing the examination results. For example, with respect to the autonomic nervous system, the examination includes a sympathetic skin response (SSR), sympathetic perspiration, and pupillary contraction.

### SUMMARY

An aspect of the present invention provides a dual-pupil measuring device that emits light beams for exciting two pupils, and records changes in the contractions of the two pupils. The dual-pupil measuring device records images of the two pupils by emitting visible light beams and invisible light beams along an optical path formed by one of a plurality of light sources, one of the eyes, an optical unit, and an imaging unit, in which the images of the two pupils before and after contraction are recorded by the imaging unit.

An aspect of the present invention provides a method for measuring two pupils. The method includes a number of steps. A first visible light beam and a first invisible light beam are emitted toward a right eye. A second visible light beam and a second invisible light beam are emitted toward a left eye. The light beams reflected from the right eye and the left eye are received by an optical unit, and the first invisible light beam and the second invisible light beam are guided to an imaging unit by the optical unit. Images of the right eye and the left eye are respectively recorded through the first invisible light and the second invisible light beam propagated from the optical unit.

In some embodiments, the first invisible light beam and the second invisible light beam have different wavelength bands. The step of guiding the first invisible light beam and the second invisible light beam to the imaging unit further includes guiding the first invisible light beam and the second invisible light beam to the same position of the imaging unit, such that the images of the right eye and the left eye are overlapped with each other.

In some embodiments, the first visible light beam and the second visible light beam are emitted simultaneously.

In some embodiments, the first visible light beam and the second visible light beam are emitted alternatingly.

In some embodiments, the first visible light beam and the second visible light beam have the same wavelength band.

In some embodiments, the method further includes a step of calculating a time difference between time points when the diameters of the pupils in the images of the right eye and the left eye start to change.

In some embodiments, the method further includes a step of adjusting a parameter of the first visible light beam and the second visible light beam and calculating a difference between the images of the right eye and the left eye. The parameter between the first visible light beam and the second visible light beam includes a parameter in wavelength, a parameter in light intensity, a parameter in light frequency, a parameter in irradiation time, a parameter in emitting-light time, or combinations thereof.

An aspect of the present invention provides a dual-pupil measuring device including a right-eye light source, a left-eye light, source, an optical unit, and an imaging unit. The right-eye light source includes a right-eye-exciting light source and a right-eye-illuminating light source. The right-eye-exciting light source is used for providing a first visible light beam toward a right eye. The right-eye-illuminating light source is used for providing a first invisible light beam toward the right eye. The left-eye light source includes a left-eye-exciting light source and a left-eye-illuminating light source. The left-eye-exciting light source is used for providing a second visible light beam toward a left eye. The left-eye-illuminating light source for providing a second invisible light beam toward the left eye. The optical unit is used for receiving and guiding the light beams reflected from the right eye and the left eye. The imaging unit is used for receiving the light beams guided by the optical unit, in which images of the right eye and the left eye are recorded by the imaging unit through the first invisible light beam and the second invisible light beam respectively.

In some embodiments, the wavelength bands of the first invisible light beam and the second invisible light beam are independent of each other. The optical unit includes a right-eye filter, a left-eye filter, and a light-combining element. The right-eye filter is used for filtering the first visible light beam and allowing the first invisible light beam to pass therethrough. The left-eye filter is used for filtering the second visible light beam and allowing the second invisible light beam to pass therethrough. The light-combining element is used for receiving the light beams propagated from the right-eye filter and the left-eye filter, in which the first invisible light beam and the second invisible light beam are guided to the same position of the imaging unit and overlapped with each other by the light-combining element.

In some embodiments, the imaging unit includes a right-eye image sensor and a left-eye image sensor. The right-eye image sensor is used for recording the image of the right eye via the first invisible light beam. The left-eye image sensor is used for recording the image of the left eye via the second invisible light beam.

An aspect of the present invention provides a method for measuring two pupils and a dual-pupil measuring device. A combination of the method and the dual-pupil measuring device operates to record and measure the changes in the contractions of the two pupils. Therefore, by the combination of the method and the dual-pupil measuring device, a difference in the changes of the contractions of the two pupils is obtained, and a neurotransmission between right and left brains can be further analyzed. Hence, an autonomic nervous system disorder can be detected and found.

### BRIEF DESCRIPTION OF THE DRAWINGS

The invention can be more fully understood by reading the following detailed description of the embodiments, with reference made to the accompanying drawings as follows:

FIG. 1 is a front view of a dual-pupil measuring device according to a first embodiment of this invention;

FIG. 2 is a schematic diagram of measuring a right eye by the dual-pupil measuring device in FIG. 1, in which an optical path used in the measurement is illustrated;

FIG. 3 is a schematic diagram of measuring a pupil using a method for measuring two pupils according to an embodiment of this invention;

FIG. 4 is a schematic diagram of measuring two pupils using a method for measuring two pupils according to an embodiment of this invention;

FIG. 5 is a graph illustrating diameters of two pupils in relation to a time of measuring the diameters of the two pupils using a method for measuring two pupils according to an embodiment of this invention;

FIG. 6A to FIG. 6C are images of two pupils corresponding to time points t₁, t₂, and t₃ in FIGS. 5; and

FIG. 7 is a front view of a dual-pupil measuring device according to a second embodiment of this invention.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

An aspect of the present invention provides a method for measuring two pupils. The method is used for recording and measuring changes in the contractions of two pupils when exciting the two pupils by light beams. As far as a reflex of the pupil is concerned, there are two types of reflexes. The first type of reflex is a direct light reflex. For example, a right-eye pupil contracts when the right eye is excited. The second reflex is an indirect light reflex. For example, a left-eye pupil contracts when a right eye (the other eye) is excited. Regardless of the type of reflex, the method for measuring two pupils of the present invention still can simultaneously record and measure changes in the contractions of the two pupils. Furthermore, when the two pupils start to contract at different times, the method for measuring two pupils of the present invention can further measure a time difference between the contractions of the two pupils.

A method for measuring two pupils of the present invention includes a number of steps. A first visible light beam and a first invisible light beam are emitted toward' a right eye. A second visible light beam and a second invisible light beam are emitted toward a left eye. The light beams reflected from the right eye and the left eye are received by an optical unit, and the first invisible light beam and the second invisible light beam are guided to an imaging unit by the optical unit. Images of the right eye and the left eye are respectively recorded through the first invisible light beam and the second invisible light beam propagated from the optical unit. In addition, in the method for measuring two pupils of the present invention, the images of the right eye and the left eye can be integrated by a dual-pupil measuring device, such that the images of the right eye and the left eye can be recorded simultaneously. The dual-pupil measuring device will be described in greater detail below.

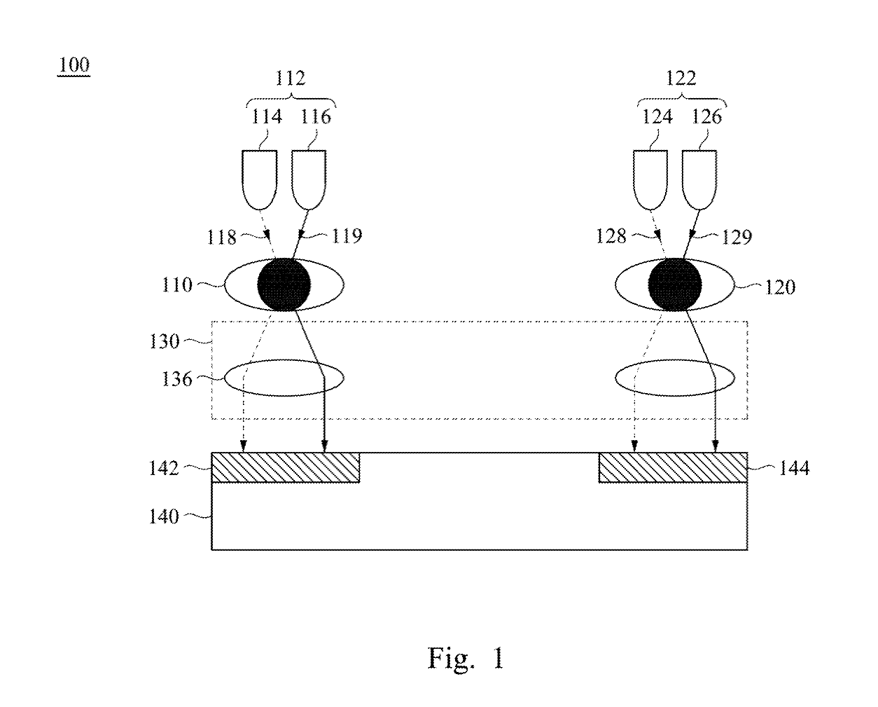

FIG. 1 is a front view of a dual-pupil measuring device according to a first embodiment of this invention. A dual-pupil measuring device 100 includes a right-eye light source 112, a left-eye light source 122, an optical unit 130, and an imaging unit 140.

The right-eye light source 112 includes a right-eye-exciting light source 114 and a right-eye-illuminating light source 116. The right-eye-exciting light source 114 is used for providing a first visible light beam 118 toward a right eye 110. The right-eye-illuminating light source 116 is used for providing a first invisible light beam 119 toward the right eye 110. The left-eye light source 122 includes a left-eye-exciting light source 124 and a left-eye-illuminating light source 126. The left-eye-exciting light source 124 is used for providing a second visible light beam 128 toward a left eye 120. The left-eye-illuminating light source 126 for providing a second invisible light beam 129 toward the left eye 120. The optical unit 130 is used for receiving and guiding the light beams reflected from the right eye 110 and the left eye 120. The imaging unit 140 is used for receiving the light beams guided by the optical unit 130, in which images of the right eye 110 and the left eye 120 are recorded by the imaging unit 140 through the first invisible light beam 119 and the second invisible light beam 129 respectively.

In addition, with respect to the right-eye-illuminating light source 116 and the left-eye-illuminating light source 126, “illuminating” refers to the fact that the imaging unit 140 can obtain the images through illuminating the right eye 110 by the first invisible light beam 119 and illuminating the left eye 120 by the second invisible light beam 129. That is, the imaging unit 140 records the images of the right eye 110 and the left eye 120 by receiving the light beams provided by the right-eye-illuminating light source 116 the left-eye-illuminating light source 126.

The aforementioned method for measuring two pupils can be performed by the dual-pupil measuring device 100 of the present embodiment, in which the dual-pupil measuring device 100 is used for emitting the light beams for exciting the two pupils and recording changes in contractions of the two pupils. In some embodiments, each of the light beams in the dual-pupil measuring device 100 travels along an optical path from one of the light sources, one of the eyes, the optical unit 130, and the imaging unit 140 in sequence. Through the dual-pupil measuring device 100 and the method for measuring two pupils, a difference between the changes in the contractions of the two pupils is obtained, such that a lag in a neurotransmission between right and left brains can be further analyzed and thereby an autonomic nervous system disorder can be detected and found.

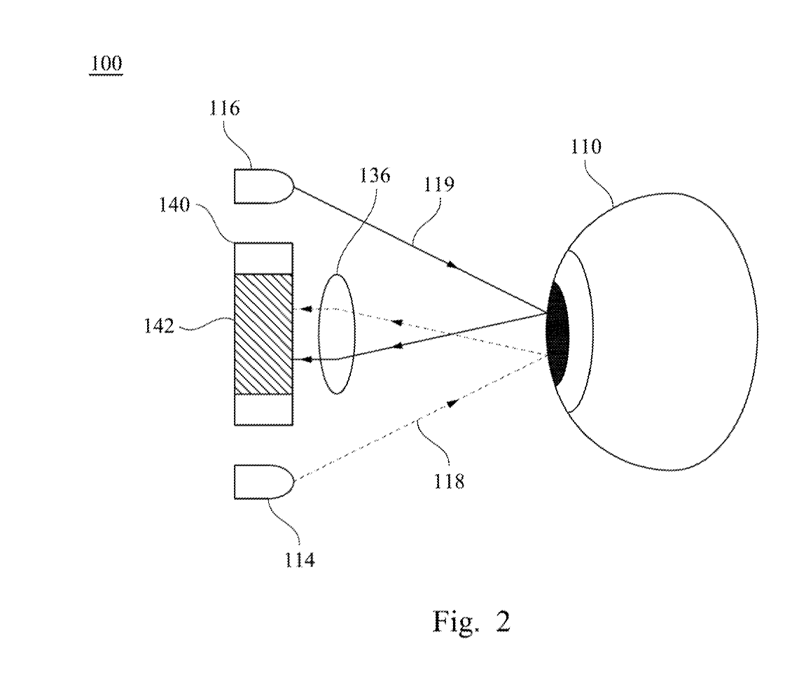

FIG. 2 is a schematic diagram of measuring a right eye by the dual-pupil measuring device 100 in FIG. 1, in which an optical path used in the measurement is illustrated. As shown in FIG. 1 and FIG. 2, in the present embodiment, the imaging unit 140 records the images of the pupil of the right eye 110 and the pupil of the left eye 120 by approximately the same optical path (one of the light sources, one of the eyes, the optical unit 130, and the imaging unit 140 in sequence). Therefore, in the description below, recording the image of the pupil of the right eye 110 is used as an example, and it should be understood that recording the image of pupil of the right eye 110 and recording the image of the pupil of the left eye 120 are approximately the same.

In this configuration, the first visible light beam 118 provided by the right-eye-exciting light source 114 and the first invisible light beam 119 provided by the right-eye-illuminating light source 116 are emitted toward the pupil of right eye 110. When the right eye 110 is excited by the first visible light beam 118, the pupil of the right eye 110 starts to contract. On the other hand, since the pupil of the right eye 110 can not detect (or perceive) the existence of the first invisible light beam 119, the first invisible light beam 119 does not affect the pupil contraction of the right eye 110.

After the first visible light beam 118 and the first invisible light beam 119 are reflected from the right eye 110, the first visible light beam 118 and the first invisible light beam 119 enter an optical lens 136 of the optical unit 130. Next, the first visible light beam 118 and the first invisible light beam 119 are guided to the imaging unit 140 by the optical lens 136. In some embodiments, the imaging unit 140 includes a right-eye image sensor 142 and a left-eye image sensor 144. The right-eye image sensor 142 is used for recording the image of the right eye 110 via the first invisible light beam 119. The left-eye image sensor 144 is used for recording the image of the left eye 120 via the second invisible light beam 129.

As shown in FIG. 2, the first visible light beam 118 and the first invisible light beam 119 enter the right-eye image sensor 142. In some embodiments, the right-eye image sensor 142 includes a near-infrared photosensitive material, in which the near-infrared photosensitive material detects a wavelength in the range from 700 nm to 2000 nm, which belongs to the near-infrared band. Correspondingly, the wavelength of each of the first invisible light beam 119 and the second invisible light beam 129 are also in the range from 700 nm to 2000 nm. In addition, since the right-eye image sensor 142 and the left-eye image sensor 144 of the imaging unit 140 are disposed separately, a measuring result is not affected by a wavelength-overlapping relationship between the first invisible light beam 119 and the second invisible light beam 129. A person having ordinary skill in the art may choose a proper relationship between the wavelengths of the first invisible light beam 119 and the second invisible light beam 129. For example, the relationship between the wavelengths of the first invisible light beam 119 and the second invisible light beam 129 is fully overlapping, partially overlapping, or non-overlapping.

Therefore, the image of the pupil of the right eye 110 can enter the right-eye image sensor 142 via the first invisible light beam 119, and then an electronic image of the pupil of the right eye 110 is generated in the imaging unit 140. However, since the wavelength of the first visible light beam 118 is out of the near-infrared band, the image recorded by the imaging unit 140 is not affected by the first visible light beam 118.

Moreover, the first visible light beam 118 is used for exciting the right eye 110, and the first invisible light beam 119 is used for illuminating the right eye 110. Therefore, the imaging unit 140 can record the image of the pupil of the right eye 110 via the first invisible light beam 119, whether the first visible light beam 118 is emitted by the right-eye-exciting light source 114 or not. That is, if an initial diameter of the pupil of the right eye 110 needs to be measured, the first invisible light beam 119 is emitted before emitting the first visible light beam 118.

As shown in FIG. 1, the right-eye light source 112 and the left-eye light source 122 are disposed separately, and the right-eye image sensor 142 and the left-eye image sensor 144 of the imaging unit 140 are also disposed separately. Therefore, recording the image of the pupil of the right eye 110 and recording the image of the pupil of the left eye 120 are also separate. In other words, the first visible light beam 118 and the second visible light beam 128 for exciting the pupils are emitted separately. In some embodiments, the first visible light beam 118 and the second visible light beam 128 are emitted simultaneously. Moreover, in some embodiments, the first visible light beam 118 and the second visible light beam 128 are emitted alternatingly.

In addition, whether the first visible light beam 118 and the second visible light beam 128 are emitted simultaneously or emitted alternatingly, the two pupils are recorded simultaneously. Here, “the two pupils are recorded simultaneously” refers to the fact that the diameters of the two pupils are recorded and measured continuously. That is, during the measurement, the two pupils can be excited by the light beams at different times or the same time, while the diameters of the two pupils are recorded and measured at the same time (or at the same time point), regardless of whether the two pupils are excited at different times or the same time. Moreover, the diameters of the two pupils are recorded and measured from the beginning to the end of the measurement. Through measuring the diameters of the two pupils simultaneously, the states of pupil contraction and the changes in pupil contraction can be observed quickly and clearly. The manner in which the light beams are emitted and the conditions of the light beams will be described in greater detail below.

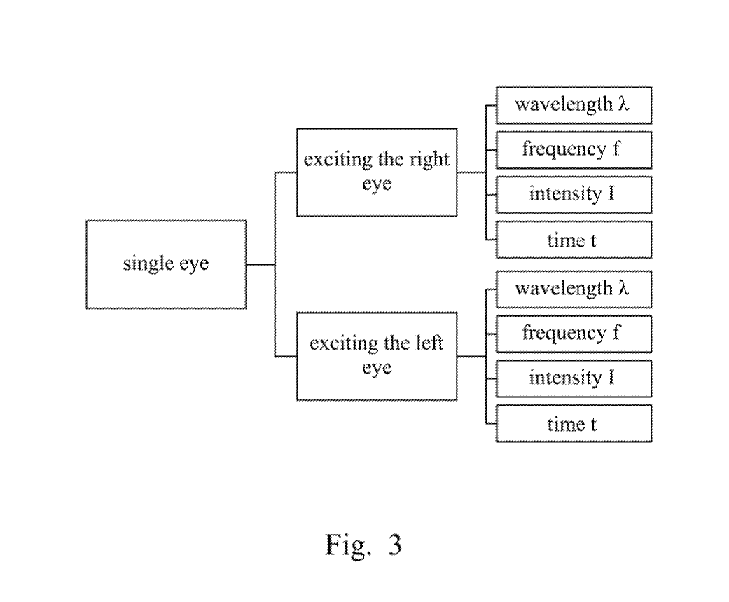

FIG. 3 is a schematic diagram of measuring a single pupil using a method for measuring two pupils according to an embodiment of this invention. As shown in FIG. 1 and FIG. 3, in the present embodiment, one of the eyes is excited by the light beam, and recording the images of the two pupils is still performed simultaneously. For example, the pupil of the right eye is excited alone, and the two pupils are recorded.

In the beginning of the measurement, the right-eye-illuminating light source 116 of the right-eye light source 112 and the left-eye-illuminating light source 126 of the left-eye light source 116 are operated, such that initial states of the two pupils can be recorded. Next, the right-eye-exciting light source 116 of the right-eye light source 112 is operated for exciting the pupil of the right eye 110 via the first visible light beam 118. Since recording the right eye 110 and recording the left eye 120 are performed simultaneously, a difference between the changes in the contractions of the two pupils can be obtained via the images of the right eye 110 and the left eye 120. On the other hand, exciting the pupil of the left eye 120 alone and recording the changes in the contractions of the two pupils may also be performed.

In measuring the single pupil of the present embodiment, parameters including wavelength λ (or wavelength band), frequency f, intensity I, and time t (irradiation time) can be taken as variables during the measurement. For instance, during the measurement involving exciting the pupil of the right eye 110 via the first visible light beam 118, the wavelength λ, frequency f, intensity I, and time t of the first visible light beam 118 can be taken as the variables. For example, assuming the wavelength of the first visible light beam 118 is the variable, the color of the first visible light beam 118 can be varied through adjusting the wavelength λ of the first visible light beam 118. Furthermore, since the human eye has different sensitivities to different colors, the examination of neurotransmission can be further analyzed by taking the wavelength as the variable.

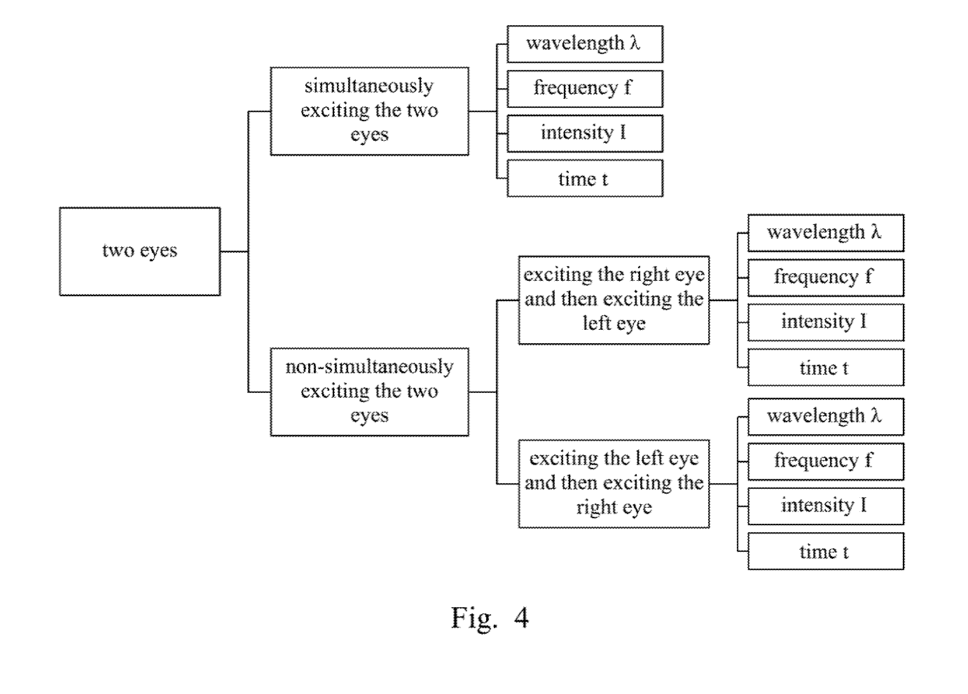

FIG. 4 is a schematic diagram of measuring two pupils using a method for measuring two pupils according to an embodiment of this invention. As shown in FIG. 1 and FIG. 4, in the present embodiment, the two eyes are excited by the light beams, in which exciting the two eyes includes simultaneously exciting and non-simultaneously exciting the two eyes.

In a measurement involving simultaneously exciting the two eyes, at the beginning of the measurement, the right-eye-illuminating light source 116 of the right-eye light source 112 and the left-eye-illuminating light source 126 of the left-eye light source 122 are operated, such that initial states of the two pupils can be recorded. Next, the right-eye-exciting light source 116 of the right-eye light source 112 and the left-eye-exciting light source 124 of the left-eye light source 122 are operated simultaneously for exciting the pupil of the right eye 110 and the pupil of the left eye 120 via the first visible light beam 118 and the second visible light beam 128 respectively. Subsequently, a difference between changes in the contractions of the two pupils is obtained by the images of the right eye 110 and the left eye 120.

In a measurement involving non-simultaneously exciting the two eyes, at the beginning of the measurement, the right-eye-illuminating light source 116 of the right-eye light source 112 and the left-eye-illuminating light source 126 of the left-eye light source 122 are operated, such that initial states of the two pupils can be recorded. Next, the right-eye-exciting light source 116 of the right-eye light source 112 is operated for exciting the pupil of the right eye 110 via the first visible light beam 118. After exciting the pupil of the right eye 110 via the first visible light beam 118, the left-eye-exciting light source 124 of the left-eye light source 122 is operated for exciting the pupil of the left eye 120 via the second visible light beam 128. That is, in the measurement involving non-simultaneously exciting the two eyes, the two pupils are respectively excited at different times, and then a difference between changes in the contractions of the two pupils is obtained via the images of the right eye 110 and the left eye 120.

Similarly, in measuring the two pupils of the present embodiment, the parameters including wavelength λ (or wavelength band), frequency f, intensity I, and time t (irradiation time) can be taken as the variables during the measurement. In some embodiments, the method for measuring two pupils further includes a step of adjusting a parameter of the first visible light beam 118 and the second visible light beam 128 and calculating a difference between the images of the right eye 110 and the left eye 120. The parameter between the first visible light beam 118 and the second visible light beam 128 includes a parameter in wavelength, a parameter in light intensity, a parameter in light frequency, a parameter in irradiation time, a parameter in emitting-light time, or combinations of these parameters. For example, through adjusting the wavelength λ, the two pupils can be excited by the first visible light beam 118 and the second visible light beam 128 using different colors, in which the first visible light beam 118 and the second visible light beam 128 may have the same of different colors.

As previously described, the method for measuring two pupils can record the images of the two pupils simultaneously with the dual-pupil measuring device, whether the measurement is performed by exciting a single eye or exciting both eyes. Hence, the states of the contractions of the two pupils in the different variables are obtained, and the difference between the changes in the contractions of the two pupils can be determined and analyzed.

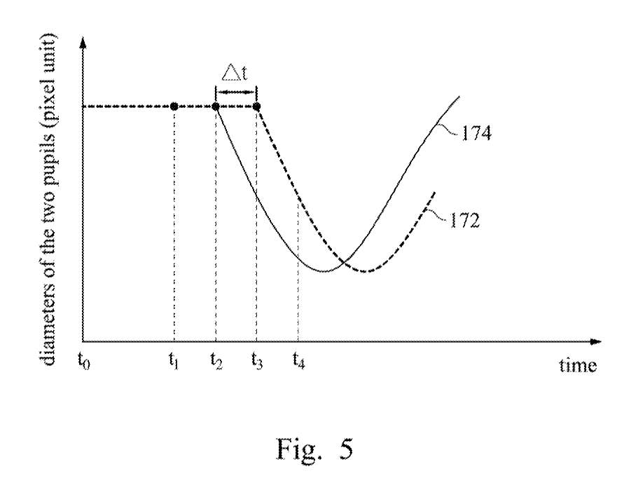

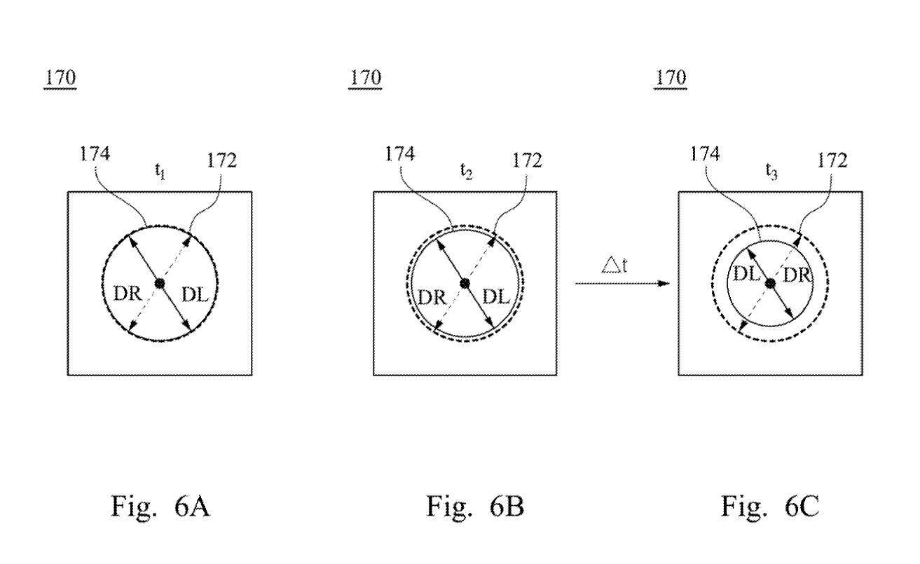

A description will now be provided of the details of the measurement involving simultaneously exciting two eyes with reference to FIG. 5 and FIGS. 6A to 6C below. FIG. 5 is a graph illustrating a relation between diameters of two pupils and a time in measuring the diameters of the two pupils using a method for measuring two pupils according to an embodiment of t his invention. In FIG. 5, the vertical axis represents the diameters of the two pupils, in which unit of the diameters of the two pupils is a pixel unit of the imaging unit 140 (see FIG. 1). Moreover, the horizontal axis represents time and includes time point t₀, time point t₁, time point t₂, time point t₃, and time point t₄, in which time point t₀ corresponds to an initial time point of the measurement. That is, the time point t₀ corresponds to the beginning of the measurement. Furthermore, in FIG. 5, the dotted line corresponds to a right-eye-pupil image 172, and the solid line corresponds to a left-eye-pupil image 174.

FIG. 6A to FIG. 6C are images of the two pupils corresponding to time point t₁, time point t₂, and time point t₃ in FIG. 5. FIG. 6A to FIG. 6C are illustrated as a superposition image 170, in which this superposition image 170 is formed by superposing the images of the two pupas recorded by the imaging unit 140 (see FIG. 1). The superposition image 170 has the right-eye-pupil image 172 (shown by the dotted line) and the left-eye-pupil image 174 (shown by the solid line). Moreover, in FIG. 6A to FIG. 6C, the right-eye-pupil image 172 has a diameter DR, and the left-eye-pupil image 174 has a diameter DL.

As shown in FIG. 1 and FIG. 5, at the beginning of the measurement (at time point t₀), the right-eye-illuminating light source 116 of the right-eye light source 112 and the left-eye-illuminating light source 126 of the left-eye light source 122 are operated. Therefore, the right-eye-pupil image 172 and the left-eye-pupil image 174 are respectively recorded by the right-eye image sensor 142 and the left-eye image sensor 144 of the imaging unit 140 via the first invisible light beam 119 and the second invisible light beam 129. In addition, since the first invisible light beam 119 and the second invisible light beam 129 cannot be detected (or perceived) by the human eye, the two pupils do not contract at this time, as shown in FIG. 6A. In FIG. 6A, the diameter DR of right-eye-pupil image 172 and the diameter DL of left-eye-pupil image 174 are approximately the same. However, FIG. 6A is a conceptual diagram, and the two pupils may have different diameters for some people.

In a period from time point t₁ to time point t₂, the right-eye-exciting light source 114 of the right-eye light source 112 and the left-eye-exciting light source 124 of the left-eye light source 122 are operated, and the right-eye-illuminating light source 116 of the right-eye light source 112 and the left-eye-illuminating light source 126 of the left-eye light source 122 keeps operating. In some embodiments, the first visible light beam 118 and the second visible light beam 128 have the same wavelength (or wavelength band). Therefore, the two pupils are excited by the first visible light beam 118 and the second visible light beam 128 under the same condition and at the same time. In this period, after the pupil of the right eye 110 and the pupil of the left eye 120 are excited by the first visible light beam 118 and the second visible light beam 128 respectively, the pupils of the right eye 110 and the left eye 120 start to contract.

In addition, under normal circumstances, since neurotransmission has a time lag, pupil contraction does not occur immediately and there is also a time lag between the contractions of the two pupils. For example, as shown in FIG. 5, although both the two pupils are excited in the period from time point t to time point t₂, the pupil of the left eye 120 starts to contract at the time point t₂, and the pupil of the right eye 110 starts to contract after a time lag when the pupil of the left eye 120 starts to contract.

At time point t₂, since the pupil of the left eye 120 contracts first, a difference between the diameters of the two pupils is correspondingly generated, as show in FIG. 6B. In FIG. 6B, the diameter DL of the left-eye-pupil image 174 is smaller than the diameter DR of the right-eye-pupil image 172, and hence a difference between the diameters of the two pupils is ascertainable from the superposition image 170.

At time point t₃, the pupil of the right eye 110 starts to contract, and hence there is a time difference Δt between the time points when the pupils of the right eye 110 and the left eye 120 start to contract. In some embodiments, the method for measuring two pupils further includes a step of calculating the time difference Δt between the time points when the diameters of the pupils in the images of the right eye 110 and the left eye 120 start to change. By the above step, the time difference Δt can be measured in a state of simultaneously exciting the dual eyes. As far as a transmission-delay in neurotransmission of right and left brains is concerned, the delay can be further analyzed via the time difference Δt.

At time point t₄, the right-eye-exciting light source 114 of the right-eye light source 112 and the left-eye-exciting light source 124 of the left-eye light source 122 are turned off simultaneously, such that the two pupils start to dilate due to the weak illumination. At this time, the right-eye-illuminating light source 116 of the right-eye light source 112 and the left-eye-illuminating light source 126 of the left-eye light source 122 still keep operating, such that the images of the two pupils can be recorded simultaneously and continuously by the imaging unit 140. Similarly, since recording the images of the two pupils is simultaneous, a difference between changes in the dilation of the two pupils and a state of the dilation of the two pupils also are still recorded and measured.

In addition it is to be noted that FIG. 5 is a conceptual diagram, and under different conditions, the sequence of the pupil contraction and pupil dilation between the right eye 110 and the left eye 120 may be different from FIG. 5, and the time difference Δt of the pupil contraction may be also different from FIG. 5.

According to the above embodiments, the two pupils can be recorded and measured simultaneously via the method for measuring two pupils of the present invention, and the difference between the changes in the two pupils may be obtained from the results of the measurement. Moreover, in some embodiments, the light beams for exciting the two pupils may have different parameters such that the measurement has different variables, in which the parameters (or variables) include wavelength λ, frequency f, intensity I, and time t.

Furthermore, corresponding to the different variables, by the above steps, the difference between the contractions of the two pupils can be recorded and measured. For example, a time difference between time points when the two eyes start to contract, a diameter difference between the two pupils before and after contracting, a time difference between the two pupils reaching a steady state after contracting, a time difference between time points when the two pupils start to dilate, a velocity difference between the two pupils in contracting, or a velocity difference between the two pupils in dilating.

Moreover, the method for measuring two pupils further includes superposing the images of the right and left eyes. In some embodiments, the first invisible light beam and the second invisible light beam have different wavelength bands. The step of guiding the first invisible light beam and the second invisible light beam to the imaging unit by the optical unit further includes guiding the first invisible light beam and the second invisible light beam to the same position of the imaging unit, such that the images of the right eye and the left eye overlap each other. Herein, “the images of the right eye and the left eye overlap each other” refers to the imaging unit directly recording an overlapping image formed by the right eye and the left eye.

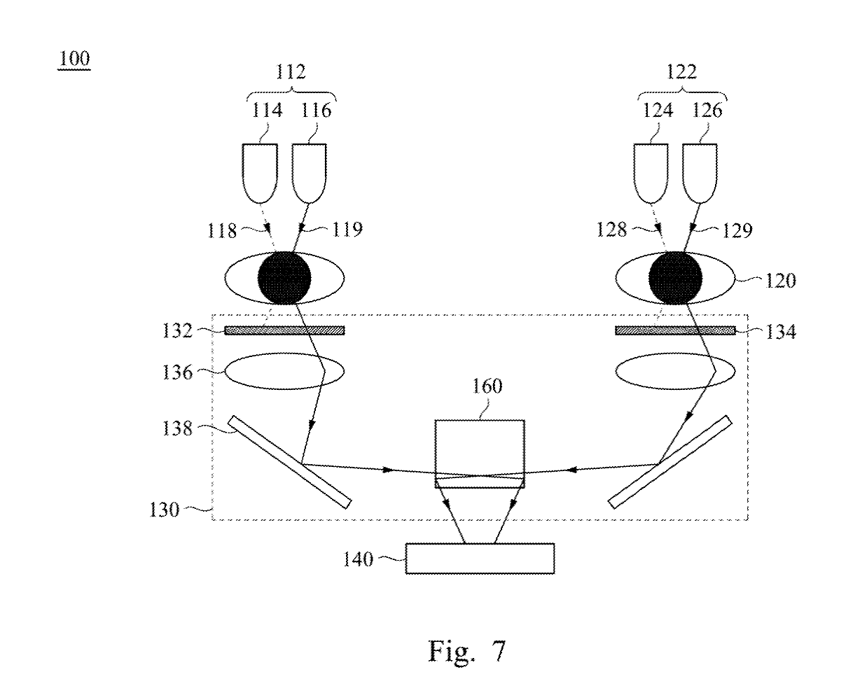

FIG. 7 is a front view of a dual-pupil measuring device according to a second embodiment of this invention. As shown in FIG. 7, a difference between the present embodiment and the first embodiment is that the imaging unit 140 directly records an overlapping image formed by the images of the right eye and the left eye.

In the present embodiment, the wavelength bands of the first invisible light beam 119 and the second invisible light beam 129 are independent of each other. The optical unit 130 includes a right-eye filter 132, a left-eye filter 134, an optical lens 136, a reflective mirror 138, and a light-combining element 160. The right-eye filter 132 is used for filtering the first visible light beam 118 and allowing the first invisible light beam 119 to pass therethrough. The left-eye filter 134 is used for filtering the second visible light beam 128 and allowing the second invisible light beam 129 to pass therethrough. The optical lens 136 and the reflective mirror 138 are used for guiding the light beams to the light-combining element 160. The light-combining element 160 is used for receiving the light beams propagated from the right-eye filter 132 and the left-eye filter 134, in which the first invisible light beam 119 and the second invisible light beam 129 are guided to the same position of the imaging unit 140 and overlapped with each other by the light-combining element 160.

In the present embodiment, the first invisible light beam 119 and the second invisible light beam 129 have different wavelength bands and are not overlapped with each other. Therefore, after the first invisible light beam 119 and the second invisible light beam 129 are superposed onto the same position of the imaging unit 140, the first invisible light beam 119 and the second invisible light beam 129 can be separately recorded due to a difference in wavelength. In addition, through the disposition of the right-eye filter 132 and the left-eye filter 134, the first visible light beam 118 and the second visible light beam 128 are not recorded by the imaging unit 140, and hence the images recorded by the imaging unit 140 are not affected by the first visible light beam 118 and the second visible light beam 128.

Since the first invisible light beam 119 and the second invisible light beam 129 are recorded by the imaging unit 140 at the same position and the same time, the imaging unit 140 can directly receive an image that is similar to the images illustrated as FIG. 6A to FIG. 6C without performing extra processes. That is, the imaging unit 140 can record the images of the two pupils and the difference between the changes in the contractions of the two pupils more precisely and instantaneously.

As previously described, the method for measuring two pupils of the present invention can record and measure the images of the two pupils with the dual-pupil measuring device. The dual-pupil measuring device emits light beams for exciting the two pupils and records the changes in the contractions of the two pupils, in which the dual-pupil measuring device records the images of the two pupils by the optical path formed by one of the light sources, one of the eyes, the optical unit, and the imaging unit. In addition, by the dual-pupil measuring device and the method for measuring two pupils, the difference between the changes in the contractions of the two pupils is obtained, such that the neurotransmission between the right and left brains can be further analyzed and thereby an autonomic nervous system disorder can be detected and found.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A method for measuring two pupils, comprising:
emitting a first visible light beam and a first invisible light beans toward a right eye;
emitting a second visible light beam and a second invisible light beam toward a left eye;
receiving the light beams reflected from the right eye and the left eye by an optical unit, and guiding the first invisible light beam and the second invisible light beam to an imaging unit; and
recording images of the right eye and the left eye respectively through the first invisible light and the second invisible light beam propagated from the optical unit.

2. The method of claim 1, wherein the first invisible light beam and the second invisible light beam have different wavelength bands, and the step of guiding the first invisible light beam and the second invisible light beam to the imaging unit further comprises:
guiding the first invisible light beam and the second invisible light beam to the same position of the imaging unit, such that the images of the right eye and the left eye are overlapped with each other.

3. The method of claim 1, wherein the first visible light beam and the second visible light beam are emitted simultaneously.

4. The method of claim 1, wherein the first visible light beam and the second visible light beam are emitted alternatingly.

5. The method of claim 1, wherein the first visible light beam and the second visible light beam have the same wavelength band.

6. The method of claim 1, comprising:
calculating a time difference between time points when the diameters of the pupils in the images of the right eye and the left eye start to change.

7. The method of claim 1, further comprising:
adjusting a parameter of the first visible light beam and the second visible light beam and calculating a difference between the images of the right eye and the left eye, wherein the parameter between the first visible light beam and the second visible light beam comprises a parameter in wavelength, a parameter in light intensity, a parameter in light frequency, a parameter in irradiation time, a parameter in emitting-light time or combinations thereof.

8. A dual-pupil measuring device, comprising:
a right-eye light source, comprising:
a right-eye-exciting light source for providing a first visible light beam toward a right eye; and
a right-eye-illuminating light source for providing a first invisible light beam toward the right eye;
a left-eye light source, comprising:
a left-eye-exciting light source for providing a second visible light beam toward a left eye; and
a left-eye-illuminating light source for providing a second invisible light beam toward the left eye;
an optical unit for receiving and guiding the light beams reflected from the right eye and the left eye; and
an imaging unit for receiving the light beams guided by the optical unit, wherein images of the right eye and the left eye are recorded by the imaging unit through the first invisible light beam and the second invisible light beam respectively.

9. The dual-pupil measuring device of claim 8, wherein the wavelength bands of the first invisible light beam and the second invisible light beam are independent of each other, and the optical unit comprises:
a right-eye filter for filtering the first visible light beam and allowing the first invisible light beam to pass therethrough;
a left-eye filter for filtering the second visible light beam and allowing the second invisible light beam to pass therethrough; and
a light-combining element for receiving the light beams propagated from the right-eye filter and the left-eye filter, wherein the first invisible light beam and the second invisible light beam are guided to the same position of the imaging unit and overlapped with each other by the light-combining element.

10. The dual-pupil measuring: device of claim 8, wherein the imaging unit comprises:
a right-eye image sensor for recording the image of the right eye via the first invisible light beam; and
a left-eye image sensor for recording the image of the left eye via the second invisible light beam.
